(19) United States
(12) Patent Application Publication
(10) Pub. No.: US 2024/0164739 A1
(43) Pub. Date: May 23, 2024

(54) SYSTEMS AND METHODS FOR ELECTRONIC STETHOSCOPE WITH ACTIVE HEADSET

(71) Applicant: Eko Devices, Inc., Oakland, CA (US)

(72) Inventors: Michael Childs, Palm Springs, CA (US); Neal Donovan, Oakland, CA (US); Dan Freschl, 94706, CA (US); Subramaniam Venkatraman, Lafayette, CA (US); Connor Landgraf, Martinez, CA (US)

(21) Appl. No.: 18/058,128

(22) Filed: Nov. 22, 2022

(51) Int. Cl. A61B 7/04; H04R 1/10; H04R 1/28

(52) U.S. Cl. CPC A61B 7/04; H04R 1/10; H04R 1/28

(57) ABSTRACT

The present description relates generally to methods and systems for an electronic stethoscope device with an active headset, including a speaker to project audio data generated by a chestpiece, the headset defining a closed back volume for the speaker. In this way, the headset provides a back volume of the speakers in order to tune the frequency response of the speakers to a target frequency mimicking the frequency produced by a conventional acoustic stethoscope.

### FIELD

The present description relates generally to methods and systems for an electronic stethoscope with an active headset.

### BACKGROUND AND SUMMARY

Auscultation, the process of listening to internal sounds of a body, has historically been performed with an acoustic stethoscope. As one example, the acoustic stethoscope may include a two-sided chestpiece attached to hollow tubing that branches to two separate earpieces. A diaphragm on one side of the chestpiece may transmit high frequency sounds to the earpieces, or a bell on the other side of the chestpiece may transmit low frequency sounds to the earpieces. However, such acoustic stethoscopes are unable to digitize sounds that can be easily analyzed and shared electronically.

In contrast, an electronic (e.g., digital) stethoscope may generate digital audio data via a chestpiece that may include components for noise amplification, digital display, sound recording, and wireless signal transmission. For example, the electronic stethoscope may transmit audio data to earpieces of the electronic stethoscope via the hollow tubing while also being able to wirelessly transmit audio data to external listening or computing devices via wireless connections such as Bluetooth® or internet.

However, the inventors herein have recognized potential issues with existing electronic stethoscope systems. Electronic stethoscopes typically inject audio signals into the hollow tubing which transmits the sound to the user's ears. However, traveling through the hollow tube modifies the frequency profile of the sound due to the mechanical filtering provided by the tube. Further, any external noise source injected into the tube—from ambient sounds or from physical contact with the tube—are directly added to the audio traveling through the tube. These two effects cause degradation in the audio quality as perceived by the user. It may be desirable to position the speaker(s) in the earpieces of the stethoscope to reduce degradation and/or modification of audio signals.

In one example, the issues described above may be addressed by an electronic stethoscope including a headset including a speaker to project audio data generated by a chestpiece, the headset defining a closed back volume for the speaker. In this way, the speakers may be positioned in the headset (e.g., in earpieces) of the electronic stethoscope rather than in the chestpiece and the hollow tubing of the headset behind the speaker may be sealed to create a closed back volume of air between the speaker and the hollow tubing. The back volume, and optional addition of a mechanical acoustic modifier positioned in the back volume, allows for optimization of the frequency response and resonance frequency of the speakers. Additionally, sealing off the back volume from the hollow tubing may mechanically isolate the hollow tubing from the speaker and the audio signal, thereby minimizing external noises that would cause degradation of the audio quality. The headset, being in electronic communication with the chestpiece via signal wiring to the speakers and possessing noise canceling features, is an active headset as opposed to a passive headset that does not possess noise canceling features as is part of a conventional acoustic stethoscope.

It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE FIGURES

FIG. 1A is a schematic drawing of an electronic stethoscope with earpieces mechanically connected to a chestpiece via a stem and an output tube.

FIG. 1B is a block diagram showing the components of the chestpiece shown in FIG. 1A.

FIG. 2 is an illustration of a headset of the electronic stethoscope.

FIG. 3 is an illustration of the headset including exploded views of earpieces and a connector of the headset.

FIG. 4 is an illustration of an exemplary assembled connector.

FIG. 5 is a cross-sectional view of a portion of the headset of the electronic stethoscope shown in FIG. 2.

FIG. 6 is a flowchart of a method for transmission and conversion of audio from an auscultated acoustic sound to a modified electronic signal through the components of the electronic stethoscope.

### DETAILED DESCRIPTION

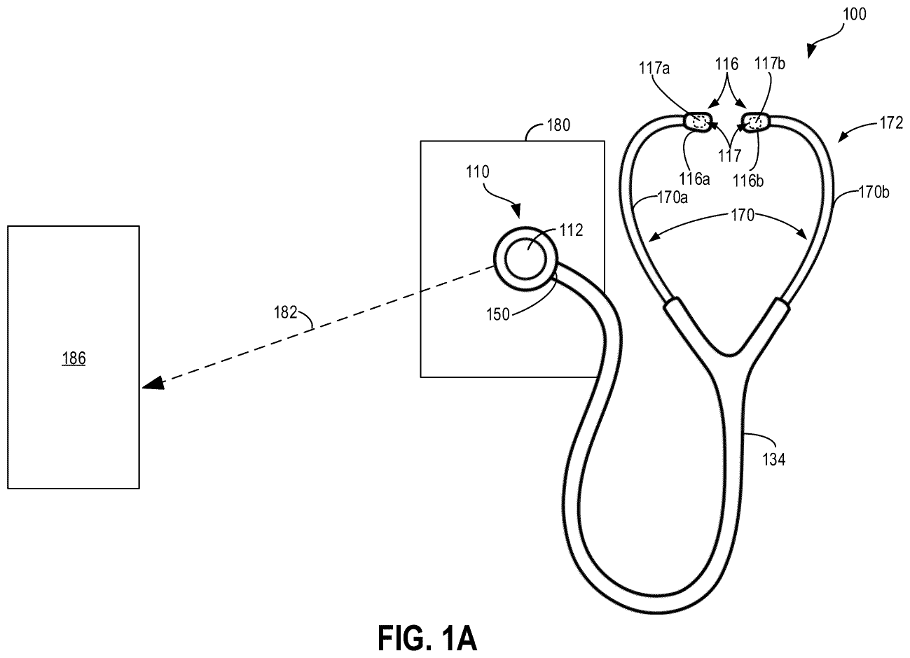

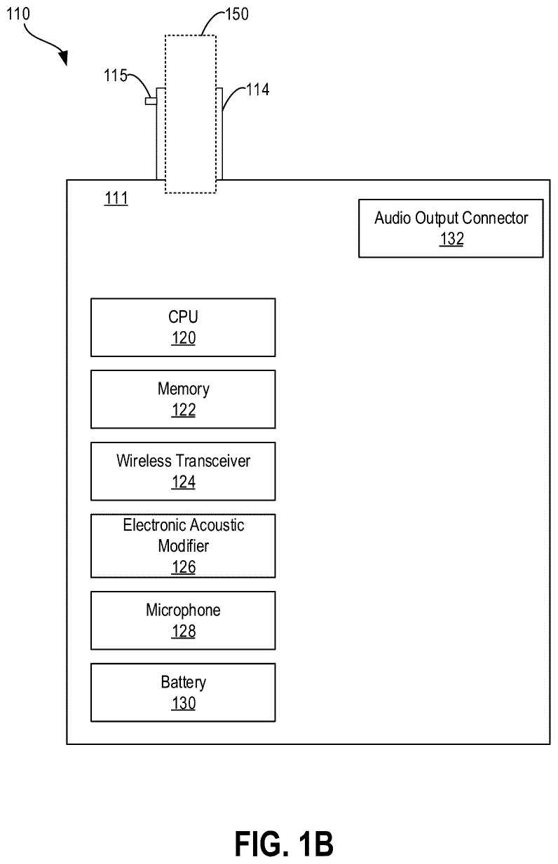

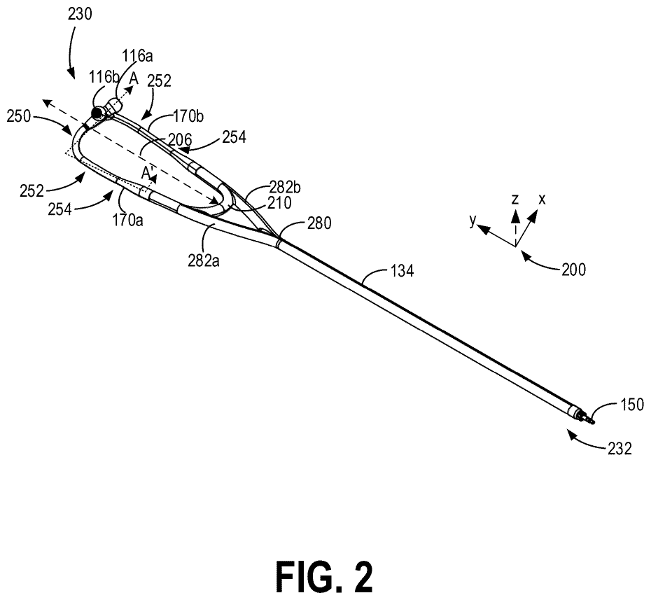

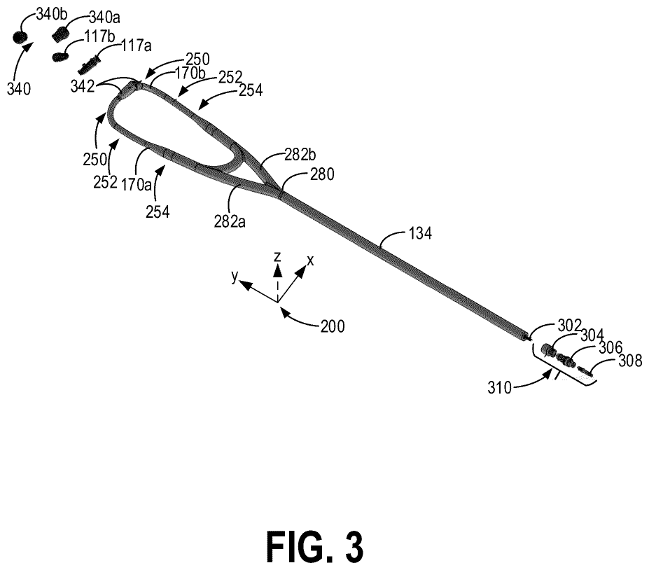

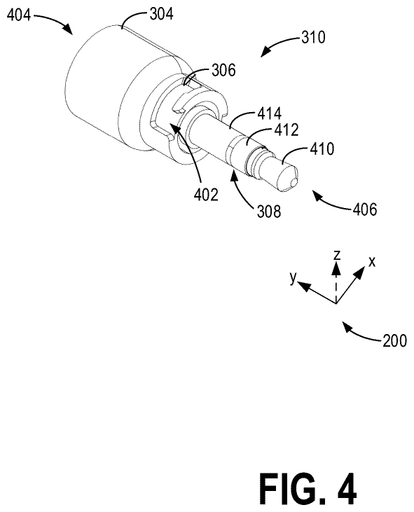

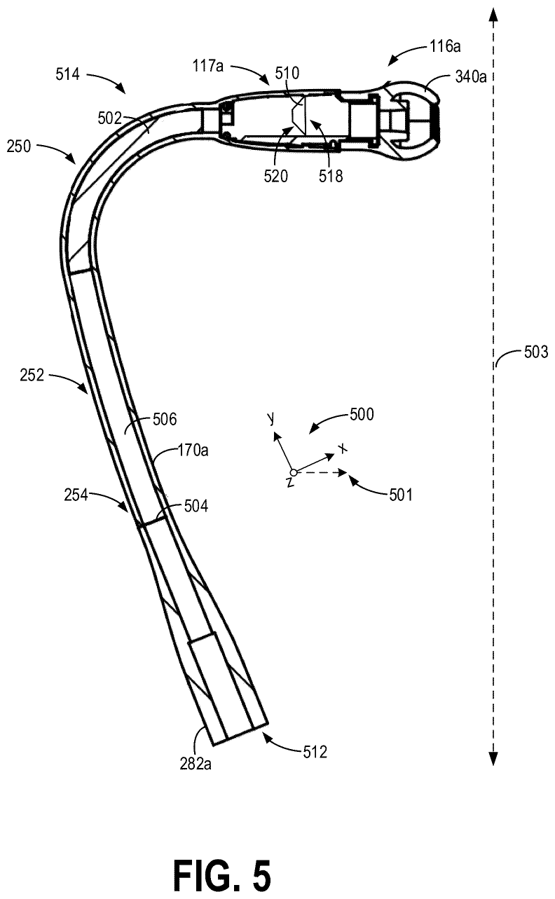

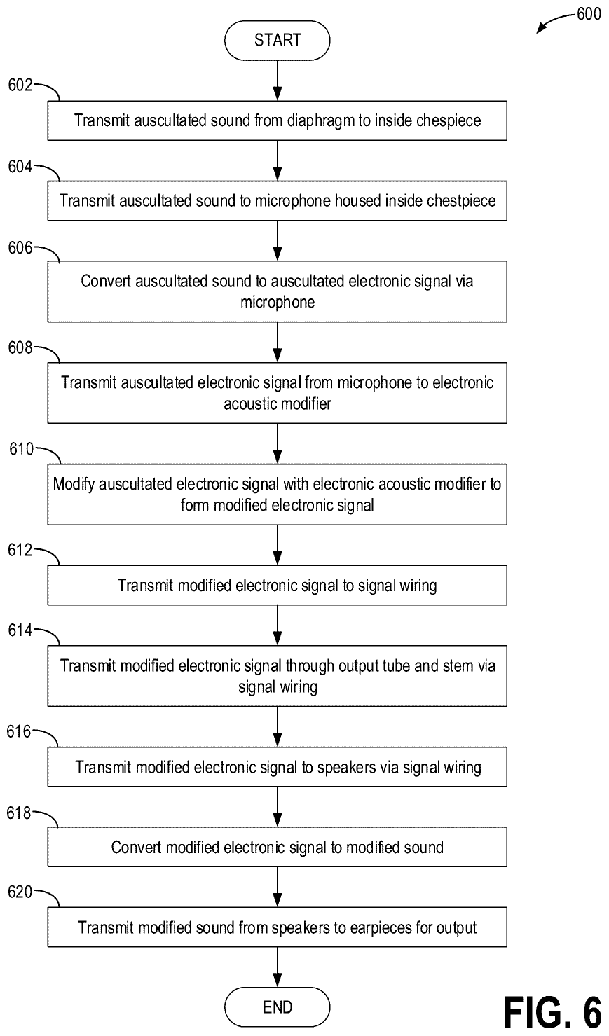

The present description relates generally to methods and systems for an electronic stethoscope with an active headset. The active headset includes speakers housed in earpieces of the headset and electronically coupled directly to a chestpiece of the electronic stethoscope, where the headset provides a back volume of the speakers in order to tune the frequency response of the speakers. For example, the digital (e.g., electronic) stethoscope may be the electronic stethoscope shown in FIG. 1A having chestpiece shown in FIG. 1B that contains electrical components of the electronic stethoscope, including components for processing and wirelessly transmitting audio data. The electronic stethoscope includes the headset shown in FIGS. 2 and 3 that includes a connector, as shown in FIG. 4, for coupling the headset to the chestpiece and providing an electronic connection between the chestpiece and the speakers of the headset. The speakers may have a frequency response that is tuned by a back volume accommodated within the headset, as shown in FIG. 5. The chestpiece may be used to record physiological sounds that are projected from the speakers, according to the method of FIG. 6.

As stated, the speakers may be housed within the earpieces of the headset, thereby providing electronic sound/signal transmission rather than acoustic sound transmission present in traditional, acoustic stethoscopes. Positioning the speakers within the earpieces may provide for a simplified design of the electronic stethoscope and may improve the quality of the audio projected to a listener via the speakers. However, physiological sounds recoded by an electronic stethoscope (e.g., heart sounds) may include lower frequency sounds, such as sounds in a range of 20-150 Hz. Further, clinicians are typically trained to use conventional acoustic stethoscopes and are therefore trained to hear the internal sounds of a patient (e.g., heart sounds, bowel sounds, lung sounds, etc.) as projected via a conventional acoustic stethoscope. Conventional acoustic stethoscopes have a distinct resonance frequency because of the frequencies produced by the sound waves traveling through the aluminum tubing of the headset. The electronic stethoscope herein described is aimed to mimic the frequencies produced by a conventional acoustic stethoscope so that it may be used by a clinician without additional training.

Outputting sound having this range of relatively low frequencies may be challenging using typical in-ear speakers (e.g., ear buds), which may have open back volumes due to small holes/ports on the earpieces of housing the speakers. For example, the sound output may not be optimal with an open back volume. Without controlling how a membrane of a speaker driver recoils with a closed back volume, distortion or unintended sounds (such as the sound of the membrane itself moving against the back of the speaker driver) may be heard by the user. This is because the membrane has to deviate far from its midpoint in order to project the low frequencies. In order to reduce the amount that the membrane has to deviate, the resonance frequency may be lowered.

The stethoscope headset disclosed herein addresses these issues with a closed back volume formed in part by the hollow tubing of the headset itself. The presence of a defined closed back volume allows for optimization of the acoustic performance of the speakers and provides a passive acoustic noise seal. An open back volume, as discussed, may not produce clear sounds at the low target frequencies herein described. In the electronic stethoscope with the active headset herein described, the closed back volume of air behind the speaker driver (and the membrane) may control the deviation of the moving parts of the speaker and provide control of the acoustic system in the 20-150 Hz range that is of interest for the electronic stethoscope. The length of the back volume may define the first resonance frequency and this length may be selected in order to tune the first resonance frequency as desired. The length and therefore total volume of the back volume may define the absolute sensitivity at low frequency. The presence and amount of a mechanical acoustic modifier such as acoustic dampening foam, which may reside within the back volume, may control the level of resonance frequency and overshoot of resonance. These three factors create a flatter frequency response at the low frequencies of interest for the electronic stethoscope compared to speakers with an open back volume.

Additionally, a binaural configuration for the digital stethoscope mimics the appearance of a conventional acoustic stethoscope, which in some circumstances may be less distracting for a patient and a clinician using the device. Further, any external noise source injected into an output tube—from ambient sounds or from physical contact with the tube—(which, in a conventional acoustic stethoscope or an electronic stethoscope that uses an output tube for acoustic sound transmission, would be heard by the user via the earpieces) may remain isolated from the audio data traveling through the signal wires to the speakers in the earpieces. This may further reduce degradation in the audio quality as perceived by the user.

Turning now to the figures, FIG. 1A shows an electronic stethoscope 100. The electronic stethoscope 100 may include a chestpiece 110 coupled to a headset 172. The headset 172 may have a binaural configuration, whereby the headset 172 includes a pair of stems 170 branching from an output tube 134 of the headset 172, with each stem coupled to a respective side of the pair of earpieces 116, one earpiece corresponding to each ear of the user. For example, the stems 170 may include a first stem 170a and a second stem 170b. The first stem 170a may connect to a first earpiece 116a and the second stem 170b may connect to a second earpiece 116b. In some examples, the first earpiece 116a may be inserted into the user's left ear and the second earpiece 116b may be inserted into the user's right ear. Each side of the headset 172 may symmetrically mirror the other.

The chestpiece 110 may be operable to generate audio data from a patient 180. In some embodiments, the chestpiece 110 may include a diaphragm 112, which is a sealed membrane with air inside that vibrates from external noises. The diaphragm 112 moves a volume of air inside the chestpiece 110 according to the vibrations caused by the external noises, which in turn creates sounds that may be recorded by a microphone of the chestpiece (described in more detail below with respect to FIG. 1B). In some examples, the chestpiece 110 may include a bell in addition to the diaphragm 112. The bell may be an open hollow cup or may include a smaller sealed membrane than the diaphragm 112, and air inside the bell may vibrate from external noises to produce acoustic pressure waves that may be recorded by the microphone. The diaphragm 112 may be used for higher frequency auscultation, such as heart beats and breath sounds, while the bell (when included) may be used for lower frequency auscultation, such as heart murmurs and bowel sounds. The chestpiece 110 may be placed on the patient 180 (e.g., a subject) by the patient 180 or by a clinician (not shown) for auscultation. The clinician or the patient 180 may listen to bodily sounds produced by the patient through the earpieces 116.

In some examples, the earpieces 116 may include speakers 117 positioned therein. The speakers 117 may be electrically couplable to the chestpiece 110. When included, the speakers 117 may be in electronic communication with the chestpiece 110 when the output tube 134 is coupled to the chestpiece 110 (e.g., via a connector 150 of the headset) and sounds received by the chestpiece 110 may be electronically transmitted to the speakers 117 in the earpieces 116 via signal wiring. As described above with reference to the binaural configuration, the earpieces 116 may include the first earpiece 116a and the second earpiece 116b. Consequently, the speakers 117 may include a first speaker 117a corresponding to the first earpiece 116a and a second speaker 117b corresponding to the second earpiece 116b. Further, the speakers 117 may be automatically powered on when the electronic stethoscope 100 is operated in an internal (e.g., wired) digital mode and automatically powered off when the electronic stethoscope 100 is operated in a wireless digital mode, as will be elaborated herein with respect to FIG. 1B. When powered on, the speakers 117 may project the audio data acquired by the chestpiece 110 through the earpieces 116 to the user's ears. The earpieces 116, housing the speakers 117, may couple to the stems 170 which in turn couple to the output tube 134, which may optionally couple to the chestpiece 110. In this way, the audio data may be transmitted from the chestpiece 110 to the speakers 117.

The chestpiece 110 may connect to other electronic devices through wireless connections. For example, the chestpiece 110 may connect to an external device 186 through a wireless connection 182. The external device 186 may be an external computing device, for example a mobile device, such as a smartphone, a tablet, a smartwatch, a laptop computer, or a personal digital assistant (PDA). Alternatively, the external device 186 may be an external computing device such as a stationary device, such as a desktop computer or server. In still other examples, the external device 186 may be included in a computing network, such as a cloud computing network. Additionally or alternatively, the external device 186 may be an external listening device and sounds received by the chestpiece 110 may be projected by the external device 186 for the patient 180 or the clinician to hear. In such embodiments, the external device 186 is an external listening device such as a speaker, headphones, earbuds, hearing aids, or another device capable of projecting sound and forming wireless connections to other devices. The external device 186 may include a processor operatively connected to memory (such as random-access memory, read-only memory, flash memory, a hard disk, etc.) as well as a communications interface for sending/receiving wired or wireless signals from a network and/or other computing devices, including the chestpiece 110. In some embodiments, the external device 186 may include a user interface (not shown), such as a display for outputting information to a user and one or more of a touchscreen, a trackball, hard keys/buttons, a keyboard, a mouse, and a trackpad for receiving user inputs. The external device 186 may operate a software application that receives the user inputs via the user interface (not shown) to adjust operation of the chestpiece 110. Upon initiation by the user, the software application may run in a background mode that facilitates an automatic connection of the external device 186 to the chestpiece 110 while reducing power consumption. By connecting wirelessly to the external device 186, the chestpiece 110 may send audio data and analysis of the audio data to the external device 186. Further configurations of different connections to various devices are possible.

Continuing to FIG. 1B, a block diagram of the components housed within the chestpiece 110 is shown. In some examples, the chestpiece 110 includes a body 111 that houses internal components, examples of which are elaborated below. The chestpiece 110 includes a computer processing unit (CPU) 120, such as a microcontroller unit (MCU), positioned within the body 111. The CPU 120 receives inputs and/or sends outputs to various electronic components that will be described further herein. In some examples, a microdevice contains the CPU 120 and some or all of the electronic and electrical components. In some arrangements, the CPU 120 and the electronic and electrical components are positioned on two or more microdevices. The CPU 120 is operatively coupled to a memory 122, which includes one or more of a non-transitory (e.g., read-only) memory, a keep alive memory, and a random-access memory. For example, the CPU 120 is configured to access and execute instructions stored in the memory 122 according to one or more routines, examples of which will be described below with respect to FIG. 3 through 6.

The chestpiece 110 may include an electronic acoustic modifier 126 in electrical communication with the CPU 120. In some examples, the electronic acoustic modifier 126 is a stand-alone device. In other examples, the electronic acoustic modifier 126 is firmware within the CPU 120. The electronic acoustic modifier 126 is configured to receive an auscultated electronic signal from a microphone 128 (e.g., the signal output by the microphone 128, which includes vibrations of the volume of air generated by the diaphragm during auscultation), modify the auscultated electronic signal to form a modified electronic signal, and transmit the modified electronic signal to the speakers 117 internal to the earpieces 116.

During the digital mode of operation when the output tube 134 is coupled to the chestpiece 110 via connector 150, the sound output from the electronic acoustic modifier 126 passes through signal wires within the output tube 134 before the sound output is transmitted to the speakers 117 in the earpieces 116 (e.g., shown in FIG. 1A) at respective ends of the headset 172.

The chestpiece 110 includes an optional audio output connector 132, such as a headphone jack or USB-type port, which can receive the modified electronic signal from the electronic acoustic modifier 126. A user may physically connect a peripheral device to the audio output connector 132. Examples of such peripheral devices include but are not limited to a computer, a cell phone, and a listening device configured to convert the modified electronic signal to sound. The audio output connector 132 may also act as a charging port in order to charge battery 130 of chestpiece 110.

In some examples, a wireless transceiver 124 is positioned in the chestpiece 110, such as within the body 111, as shown. In some examples, the wireless transceiver 124 may be included in a circuit board, such as a printed circuit board (PCB) that may also include one or more electronic components, such as the microphone 128 and the CPU 120. The wireless transceiver 124 is in electrical communication with the electronic acoustic modifier 126. The wireless transceiver 124 is configured to receive the modified electronic signal from the electronic acoustic modifier 126, convert the modified electronic signal to a modified wireless signal, and wirelessly transmit the modified wireless signal from the chestpiece to an external device, such as the external device 186 shown in FIG. 1A. The wireless transceiver 124 may use any appropriate communication types and protocol, such as television, cellular phone, Wi-Fi, satellite, two-way radio, infrared, short-range microwave signals, IEEE 802.11 compliant radio signals, Bluetooth®, or Low Energy Bluetooth (BLE). In some examples, the wireless transceiver 124 may be configured to pair directly to the external device 186, such as an external listening device and/or an external computing device. Alternatively, the wireless transceiver 124 may communicate data to the external device 186 through an intermediary device, such as a wireless router maintaining a local area network (WLAN) or through a connection to the internet. The wireless transceiver 124 may also be configured to receive signals from one or more peripheral devices, including the external device 186 shown in FIG. 1A. In some examples, the wireless transceiver 124 is in electrical communication with the microphone 128, and can wirelessly transmit the auscultated electronic signal to the external device 186 via the electronic acoustic modifier 126. In some examples, the chestpiece 110 may include a second wireless transceiver that may thereby allow the chestpiece 110 to establish two separate wireless connections with external devices.

It may be understood that sound may be projected via the speakers 117 positioned in the earpieces 116 and also transmitted via the wireless transceiver 124 at the same time. For example, a user (e.g., a clinician or the patient 180) may listen to physiological sounds while placing the electronic stethoscope 100 on the patient 180 via the earpieces 116 while one or more remote clinicians listen simultaneously via the external device 186.

In some examples, the chestpiece 110 may include a second microphone facing the external environment. The second microphone (not shown) may be configured to detect noise from the external environment and to convert the noise into an electronic noise signal. In some examples, one or both of the microphone 128 and the second microphone is a micro-electrical-mechanical system (MEMS) microphone, an electret microphone, or a piezoelectric microphone. When such a second microphone is included in the chestpiece 110, the electronic acoustic modifier 126 is configured to receive the electronic noise signal from the second microphone and to use the electronic noise signal, for example, as part of active noise cancellation, in modifying the auscultated electronic signal to form the modified electronic signal. The second microphone may sense audio from the environment via a port that couples with the environment. The second microphone and the port to the second microphone may be positioned such that they are mechanically isolated from the volume of air carrying the auscultated signal (e.g., vibrated via the diaphragm 112), the speakers 117, and the microphone 128. The mechanical isolation of the second microphone reduces coupling of the auscultation sounds that are present within the volume of air, transmitted to the microphone 128, and/or output from the speakers 117. As a result, unwanted feedback of auscultation sounds is reduced, noise cancellation processing is increased, and auscultation sound output clarity and specificity is increased.

Examples of the kinds of electronic signal modifications that may be performed using the electronic acoustic modifier 126 include, but are not limited to, active noise cancellation, noise reduction (NR), and upward or downward expansion. In an exemplary embodiment of the active noise cancellation, the electronic acoustic modifier 126 receives the electronic noise signal from the second microphone and reduces the amplitude of or removes the noise component from the auscultated electronic signal received from the microphone 128, thus increasing a quality of the modified electronic signal. NR refers to techniques which may reduce the noise portion of the modified electronic signal through the use of temporal, spectral or statistical differences between the auscultated electronic signal and the electronic noise signal. A downward expander can reduce the gain on a signal when the amplitude of a signal is below a pre-set threshold. In some examples, the gain is reduced to zero. Any gain reduction may minimize noise detection when the chestpiece 110 is held against the air.

In some examples, the second microphone can detect that the microphone 128 is recording sounds from “open air,” such as when the chestpiece 110 is held against the air (e.g., not against a patient body), by comparing the signals coming from the two microphones. If the signals are highly correlated, the sounds that would otherwise be transmitted to the speakers 117 and/or the external listening device may be suppressed. This would prevent amplification of sounds when the chestpiece 110 is not on a patient and could prevent accidental exposure to undesirable amplified sounds from such things as sirens, speech, doors closing, etc. If the two microphones detect significantly different sounds, it is an indication that the chestpiece 110 may be on a surface intended to be auscultated, and amplification could be employed.

It should be understood that, in describing electrical communication, the phrase, “A is in electrical communication with B,” describes both direct electrical communication from A and B or from B and A and also electrical communication that goes between A to B through the CPU 120, (e.g., from A to the CPU 120 to B and from B to the CPU 120 to A).

Chestpiece 110 may further include a battery 130. The battery 130 may be a disposable battery or a rechargeable battery. If the battery 130 is a disposable battery, the outside of the chestpiece may include a door (not shown) through which the battery 130 can be changed. If the battery 130 is a rechargeable battery, the outside of the chestpiece may include a charging port (as explained above) through which the battery 130 can be charged. Alternatively, the battery 130 may be charged wirelessly. The battery 130 is configured to supply power to the electronic components of the chestpiece, including, but not limited to, the microphone 128, the electronic acoustic modifier 126, the second microphone (when included), the speakers 117, the CPU 120, and the wireless transceiver 124.

Chestpiece 110 may also include one or more display outputs (not shown) positioned on an exterior of the chestpiece 110, such as indicator lights. In some examples, a display screen may be included on the chestpiece, with the displays screen configured to display text and/or images may also be included as a display output. The indicator lights and/or the display screen may provide information about the state of the electronic stethoscope 100 and/or provide information about the condition of the patient.

In some examples, the chestpiece 110 includes one or more devices to provide audio indicator signals (not shown) to provide sounds, such as beeps or verbal language, to indicate device operation status and/or information about the condition of the patient. In some examples, the volume of the audio indicator can be adjusted or turned off through user inputs.

In some examples, the body 111 of the chestpiece 110 may be connected to the output tube 134 shown in FIG. 1A via the connector 150 of the headset 172. The connector 150 may be configured to be positioned within a corresponding connector 114 of the chestpiece 110. The connector 150 may be a bayonet connector, for example, or another type of coupler connected to a jack (e.g., headphone jack) that enables electrical connection between the signal wires in the output tube 134 and the electrical components of the chestpiece 110 (e.g., the electronic acoustic modifier 126). Thus, the connector 114 may house connector 150 in order to mechanically and electrically couple the earpieces 116 (and, hence, speakers 117) to the chestpiece 110. The connector 150 may be integrated with (e.g., part of) the output tube 134 or may be a separate fitting. Further, in examples where the connector 150 is a bayonet connector, the chestpiece 110 may include a radial pin 115 (e.g., positioned on an exterior of chestpiece 110, such as on connector 114) configured to be accommodated in a slot of the bayonet connector, as will be explained in more detail below.

The second microphone, if present, and the port to the second microphone may be positioned such that they are mechanically isolated from the speakers 117, a back volume (e.g., back volume 506 as depicted in FIG. 5), and the microphone 128. The mechanical isolation of the second microphone reduces coupling of the auscultation sounds that are present within the airway, transmitted to the microphone 128, and/or output from the speakers 117. As a result, unwanted feedback of auscultation sounds is reduced, noise cancellation processing is increased, and auscultation sound output clarity and specificity is increased.

Turning now to FIG. 2, an isolated perspective view of the headset of the electronic stethoscope 100 is shown. As previously described with reference to FIG. 1A, the electronic stethoscope 100 may include the headset 172 including earpieces 116 and stems 170. The headset 172 may also include output tube 134 and the connector 150. As described previously, the headset 172 has a binaural configuration and thus includes two symmetrically mirrored sides (e.g., a left side and a right side). The components with symmetric sides include the earpieces 116, the speakers 117, the stems 170, and portions of the output tube 134. For example, the stems 170 include both the first stem 170a (e.g., on the left side) and the second stem 170b (e.g., on the right side).

Each of the stems 170 may include a top section 250 that may be curved, transitioning from a vertical orientation aligning with the y-axis of coordinate system 200 to a more relatively horizontal orientation with respect to coordinate system 200. The end of the top section 250 of the stems 170 that is towards a midline 206 may be coupled to the earpieces 116 so that the earpieces 116 are oriented to more readily fit into the user's ears. Each of the stems 170 may include a middle section 252 that may be more vertical than the top section 250 of the stems 170 (which as discussed, curves from a vertical orientation towards a relatively more horizontal orientation) and the middle section 252 of the stems 170 may taper at an angle towards the midline 206 so that the axes on which the two sides of the stems 170 are aligned may eventually intersect, as shown in FIG. 2. In some examples, the stems 170 may be constructed of aluminum.

The stems 170 may be coupled to the output tube 134. For example, the output tube 134 may branch at branch point 280 into a first tube section 282a and a second tube section 282b. The first tube section 282a may couple to a first stem 170a (e.g., the left side of the stems 170) which is connected to the first earpiece 116a and the second tube section 282b may couple to a second stem 170b (e.g., the right side of the stems 170) which is connected to the second earpiece 116b. The first earpiece 116a (e.g., the left side of the earpieces 116) may be adapted to be inserted into the left ear of the user and the second earpiece 116b (e.g., the right side of the earpieces 116) may be adapted to be inserted into the right ear of the user.

In some examples, the output tube 134 includes a cross support 210 positioned vertically above the branch point 280 of the output tube 134, as depicted in FIG. 2. In some examples, the output tube 134 may be constructed of vinyl tubing, insofar that it is more malleable than the stems 170, which as stated above in some embodiments may be constructed of aluminum. Each tube section of the output tube 134 that couple to a respective stem of the stems 170 may taper inward towards the midline 206, creating a V shape as they meet at branch point 280. The output tube 134 may then extend as a single tube between the branch point 280 and the connector 150, so that the V shape becomes a Y shape. The bottom of the output tube 134, which may be closer to a bottom end 232 of the electronic stethoscope 100 than to a top end 230, may be coupled to the connector 150. The connector 150 may optionally couple to the chestpiece 110 via connector 114.

The positions of the components discussed herein with respect to the y-axis of coordinate system 200 may be as follows: the earpieces 116 may be positioned above the stems 170, the output tube 134, and the connector 150; the stems 170 may be positioned above the output tube 134 and the connector 150 but below the earpieces 116; the output tube 134 may be positioned above the connector 150 but below the earpieces 116 and the stems 170; and the connector 150 may be positioned below the earpieces 116, the stems 170, and the output tube 134.

Turning now to FIG. 3, another isolated perspective view of the electronic stethoscope 100 is shown. FIG. 3 specifically shows a partially exploded view of the electronic stethoscope 100 in which multiple components are disconnected from each other. For example, FIG. 3 depicts the earpieces 116 as disconnected, including the speakers 117 removed from the earpieces 116 and ear tips 340 disconnected from a body 342 of the earpieces 116. In some examples, the connector 150 may be a bayonet connector 310. Additionally, in the view depicted in FIG. 3, the bayonet connector 310 is disconnected from the output tube 134 and the components included in the bayonet connector 310 are depicted as disconnected.

Each side of the pair of earpieces 116 may be comprised of ear tips 340 and a body 342. The ear tips 340 are configured to be inserted into the user's ears during operation of the electronic stethoscope 100. Each body 342 of the earpieces 116 may be configured to connect to a respective stem of stems 170. Each speaker of speakers 117 may be housed within a respective body 342 of the earpieces 116. The modified sound produced by the speakers 117 may transmit through the earpieces 116 to the user's ears via the ear tips 340. Each respective side of the pair of earpieces 116, the first earpiece 116a and the second earpiece 116b, includes a respective ear tip of the pair of ear tips 340, e.g., a first ear tip 340a and a second ear tip 340b, respectively.

During conditions in which the output tube 134 is connected to the chestpiece 110 via the connector 150, the modified electronic signal from the electronic acoustic modifier 126 may be transmitted electrically via signal wires 302 that are threaded through the output tube 134 to the speakers 117 positioned at the respective ends of the headset 172. In some embodiments, the output tube 134 may be hollow, as previously described, thereby allowing the signal wires 302 to be threaded through the hollow inside of the output tube 134. The signal wires 302 may be positioned within the output tube 134 such that no part of the signal wires 302 is visible from an outside viewing perspective during conditions in which the output tube 134 is coupled to the connector 150, which may be coupled to the chestpiece 110. The signal wires 302 may be in direct electric connection with the speakers 117 located within the earpieces 116 and the chestpiece 110 insofar that the modified electronic signal transmitted to the speakers 117 originates from the acoustic input to the chestpiece 110 that is then transmitted into and modified/converted within the chestpiece 110.

The output tube 134 and hence the headset 172 may be configured to couple to the chestpiece via the connector 150, which may be a suitable type of connection mechanism. For example, the headset 172 may couple to the chestpiece 110 via a twist-lock mechanism. The twist-lock mechanism may include a bayonet connector 310 comprising a receptor on the headset 172 and a radial pin on the chestpiece 110 (e.g., radial pin 115). The receptor may include an L-shaped slot configured to accommodate the radial pin and thereby hold the headset 172 rigidly against the chestpiece 110. The radial pin may slide into an opening of the L-shaped slot and upon a suitable rotation of the headset receptor or chestpiece radial pin (e.g., less than one full rotation, such as a quarter turn), the radial pin may be moved into a locked position that is configured to hold the headset 172 at a fixed orientation relative to the chestpiece. The radial pin may be unlocked from the receptor by turning the receptor or the chestpiece 110 in the opposite direction, and thus the twist-lock mechanism may be a releasable connection mechanism. The twist-lock mechanism may further provide electrical contact between the headset and the chestpiece via a TRS jack that extends out from the receptor and is configured to establish electrical contact with components of the chestpiece 110, as explained in more detail below.

The bayonet connector 310 may include a bayonet fitting 304 (e.g., which may couple a bayonet connecting feature 306 to the headset), the bayonet connecting feature 306 (e.g., the receptor), and a jack plug 308 (e.g., the TRS jack). The components of the bayonet connector 310 are shown in FIG. 3 in an unassembled state. While a bayonet connector is shown herein, it is to be appreciated that a different type of connector may be used without departing from the scope of this disclosure. Further, in some examples, the jack plug 308 may be included without the bayonet fitting and connecting feature.

Turning now to FIG. 4, the bayonet connector 310 in an assembled state is depicted. As previously described, the bayonet connector 310 may include the bayonet fitting 304, the bayonet connecting feature 306, and the jack plug 308. In terms of orientation, when the bayonet connector 310 is in an assembled state as depicted in FIG. 4, a top end 404 of the bayonet fitting 304 may be coupled to a bottom end of the output tube 134 (e.g., near bottom end 232 of FIG. 2) and a bottom end 406 of the jack plug 308 may couple to the chestpiece 110, with the bayonet connecting feature 306 seated therebetween.

The bayonet fitting 304 may be coupled to or otherwise include the jack plug 308 (e.g., the jack plug 308 may extend through an opening in the bayonet fitting 304 and the bayonet connecting feature 306). The bayonet connecting feature 306 may include an L-shaped slot 402 configured to accommodate the radial pin 115 on the chestpiece 110. The headset 172 may be coupled to the chestpiece 110 via insertion of the radial pin 115 into an opening of the L-shaped slot 402 and rotation of the chestpiece 110/radial pin 115 (or rotation of the headset 172/bayonet connecting feature 306) into a locked position. The L-shaped slot 402 may be sized so that the locked position is reached with less than one full rotation of the chestpiece 110, such as a quarter turn. During conditions in which the connector 150 (e.g., the bayonet connector 310) is coupled to the chestpiece 110, the bottom end of the jack plug 308 may be inserted into the corresponding connector 114 of the chestpiece 110.

The L-shaped slot 402 may comprise an opening that extends in the clockwise direction (e.g., to form an L shape) with a closed edge at the bottom end 406 such that when the radial pin is rotated clockwise within the L-shaped slot 402, the radial pin may not be pulled out of the L-shaped slot 402 along the axis of the bayonet connector 310 (e.g., parallel to the y-axis of coordinate system 200). Twist-lock mechanisms, such as bayonet connector 310 in some embodiments, may apply a clamping force and may provide a detent in a locked position that may resist accidental opening.

As stated, the jack plug 308 may be a 3.5 mm TRS jack plug. In such embodiments, the jack plug 308 may have three conductors—a tip 410, a ring 412, and a sleeve 414—that may enable a balanced audio signal to be transmitted from the chestpiece 110 to the signal wires 302. The tip 410 may have a positive wire and the ring 412 may have a negative wire. When the modified electronic signal passes through the jack plug 308, both the positive wire of the tip 410 and the negative wire of the ring 412 may gather any noise or interference signals, but due to the opposite polarities (positive vs negative), the noise or interference may be canceled out. Being able to cancel out any noise or interference may help to produce a clear auscultated electronic signal.

The bayonet fitting 304 may facilitate coupling of to the output tube 134 which houses the signal wires 302 to the chestpiece 110. During conditions in which the output tube 134 and the signal wires 302 are coupled to the chestpiece 110, an electrical connection between the signal wires 302 and the chestpiece 110 via the jack plug 308 may be established. When such an electrical connection is established and the electrical components of the chestpiece 110 are powered on (e.g., activated), the signal wires 302 and the speakers 117 may also be powered on.

In some embodiments in which the jack plug 308 is a 3.5 mm TRS jack plug, the connector 114 may be configured to couple to the jack plug 308 at each of the conductors (tip 410, ring 412, and sleeve 414). The CPU 120 may detect whether the connector 150 is physically coupled to the body 111 of the chestpiece 110.

Continuing on to FIG. 5, a cross-sectional view of a segment of the headset 172 from lines A to A′ of FIG. 2 is shown, depicting the first stem 170a of the stems 170 and the first earpiece 116a of the earpieces 116. FIG. 5 depicts one half of the headset 172, demonstrating one of the two symmetrically mirrored sides of a binaural configuration (for example, the left side) of the headset 172, including the first stem 170a and the first earpiece 116a that may be inserted into the user's ear (for example, the left ear). The following descriptions may also apply to the opposite symmetric side of these components. A midline 503 may define the midpoint between the two sides of the headset 172. The first speaker 117a may be housed inside the first earpiece 116a. Each speaker may include a driver, and thus as shown the first speaker 117a includes a speaker driver 510. The speakers 117 may be positioned in an orientation within the interior of the earpieces 116 in order to produce a modified sound within a certain frequency range and amplitude that is ideal for the electronic stethoscope 100.

The cross-sectional view of the headset 172 shows a back volume 506 that is formed within the first stem 170a. The back volume 506 is the volume of air that is exposed to the backside 520 of the speaker driver 510. The back volume 506 may be positioned between the backside 520 of the speaker driver 510 and a stem seal 504. A frontside 518 of the speaker driver 510 may be opposite the backside 520 and the frontside 518 may be closer to the first ear tip 340a. A second speaker driver (not pictured) may be positioned within the second earpiece 116b. A second closed back volume (not pictured) may be formed between the backside of the second speaker driver and a second stem seal (not pictured) within the second stem 170b of the right side of the headset 172 (where, as noted, the first speaker driver corresponds to the left side of the headset 172).

A back volume is defined as the empty air space behind and around a speaker (e.g., speakers 117). The back volume 506 allows for the first speaker 117a to have a limited amount of air to push against and to prevent the speaker from being overdriven. The overall length of the back volume 506 may dictate the resonance frequency of the first speaker 117a. The total volume of the back volume 506 may dictate the absolute sensitivity of the first speaker 117a at low frequencies. An increase in the amount of back volume 506 may lower the possible frequency response and may increase sound pressure level at low frequencies increasing usable bandwidth.

As described above, FIG. 5 depicts a cross-sectional view of one side of the headset 172 wherein the side not shown symmetrically mirrors the side shown. The back volume 506 is consequently one of two closed back volumes (e.g., the first closed back volume) of the headset 172, in this example the back volume 506 is the left back volume, corresponding to the first stem 170a (e.g., the left side of the stems 170). The first back volume (e.g., back volume 506) may be formed within a hollow region of the first stem 170a and the second back volume may be formed within a second hollow region of the second stem 170b.

As mentioned, the output tube 134 and the first stem 170a may each be hollow and/or include a hollow central core extending the entire length of the output tube 134 and the first stem 170a. The hollow region may accommodate the signal wires 302 (e.g., two signal wires may be included in the output tube 134, and one of the signal wires may be accommodated in the first stem 170a while the other signal wire may be accommodated in the second stem 170b). The hollow region of the first stem 170a may extend to the distal end of the first stem 170a where the first stem 170a couples to the first earpiece 116a. Further, at the location where the first earpiece 116a couples to the first stem 170a, the first earpiece 116a may be hollow or include a passageway that leads to the backside 520 of the speaker driver 510. Collectively, the hollow portion or passageway of the first earpiece 116a and the hollow region of at least a portion of the first stem 170a forms the back volume 506 for the first speaker 117a.

The stem seal 504 (e.g., a cap or a plug) may be positioned within the hollow region of the first stem 170a, thereby defining the size of the back volume 506, specifically the length of the back volume 506 within the first stem 170a. The back volume 506 may start at the backside 520 of the speaker driver 510 and may terminate at the stem seal 504. The stem seal 504 may mechanically isolate the first stem 170a from the output tube 134. The stem seal 504 may be positioned at a selected location along the vertical axis of the first stem 170a in order to provide a target length and target volume of the back volume 506. The position of the stem seal 504 may be selected to produce a desired frequency response of the first speaker 117a, such as defining low frequency sound pressure level and flatness of the frequency response in the low frequency range that includes low audio frequencies typically produced by the heart and lungs (e.g., 20-150 Hz) in order to mimic the frequencies projected in a conventional acoustic stethoscope. The frequency response may be a measure of the magnitude of the output of a speaker compared to its input, as a function of frequency. In this way, the frequency response describes how accurately a speaker reproduces each frequency of an audio signal, in terms of amplitude. An ideal frequency response would be neutral (e.g., flat) across all desired frequencies, such that a speaker with a neutral frequency response would output a signal that matches the input signal, for each frequency. However, most speakers, and in particular in-ear speakers, do not produce a neutral frequency response due to packaging constraints that limit the size, position, etc., of speaker components such as the driver. Conventional in-ear speakers may exhibit a frequency response that is not flat in the low frequency range. For example, a conventional in-ear speaker may output low frequencies (e.g., below 150 Hz) at a fraction of the input amplitude for the low frequencies while outputting mid-range frequencies at approximately the same amplitude as the input amplitude for the mid-range frequencies. The curved frequency response for the low-range frequencies seen in conventional in-ear speakers may be particularly acute for very low frequencies (e.g., 20-40 Hz), which may be output at 25-50% of the input amplitude.

By including a closed back volume that extends into the stem of headset, behind the speaker drive, the headset described herein may flatten the frequency response in the low frequency range (e.g., 20-40 Hz, 20-100 Hz, 20-150 Hz), which may allow the low frequencies characteristic of physiological sounds projected in conventional acoustic stethoscopes to be replicated accurately in wired, in-ear speakers. In some examples, the closed back volume in the stem may provide a frequency response in the low frequency range (e.g., in the range of 20-150 Hz) that changes by less than a threshold amount across the low frequency range, such as output amplitude as a function of frequency that changes by less than 20%, by less than 10%, or by less than 5% across the low frequency range. As described above, the frequency response, resonance frequency, and sound level output by the speakers of the headset described herein may be dictated by the volume/length of the back volume, which is controlled by the position of the stem seal 504. The frequency response may be further tuned by a mechanical acoustic modifier optionally positioned within the back volume, which is explained in more detail below.

As mentioned above, the first stem 170a, sealed off from the output tube 134 by the stem seal 504, may define the volume of the back volume 506. A bottom end (e.g., closer to a bottom end 512 of the headset 172 than to a top end 514) of the back volume 506 is at the location of the stem seal 504 and a top end (e.g., closer to the top end 514 of the headset 172 than to the bottom end 512) of the back volume is at a backside 520 of the speaker driver 510. The frontside 518 of the speaker driver 510 may be closer to the midline 503 than the backside 520 of the speaker driver 510.

As described previously with reference to FIG. 3, each earpiece includes an ear tip of the pair of ear tips 340, and thus as shown the first earpiece 116a may include the first ear tip 340a. The first ear tip 340a may be positioned at the distal end of the first stem 170a, e.g., more towards the midline 503 of the headset 172 than the first speaker 117a. The first speaker 117a may be positioned more towards the midline 503 of the headset 172 than the first stem 170a but may be further from the midline 503 of the headset 172 than the first ear tip 340a. The first stem 170a may be positioned further from the midline 503 of the headset 172 than the first speaker 117a and may extend relatively horizontally away from the midline 503 until the first stem 170a curves at a suitable angle (e.g., in a range of 60-90 degrees) into a relatively vertical position. A y-axis of coordinate system 500 may correspond to a vertical axis (e.g., parallel to gravity) and an x-axis of coordinate system 500 may correspond to a horizontal axis. The angle between the relatively horizontal portion of the first stem 170a and the relatively vertical portion of the first stem 170a may be less than 90 degrees. The relatively vertical position of the first stem 170a may align with y-axis of coordinate system 500. The relatively horizontal position of the first stem 170a may align with a vector 501 of coordinate system 500. Vector 501 may have a smaller vector angle with respect to the x-axis of coordinate system 500 than to the y-axis of coordinate system 500 and consequently vector 501 may be considered relatively horizontal with respect to the y-axis.

Each back volume (e.g., back volume 506) may include a mechanical acoustic modifier within the respective stem (e.g., first stem 170a). For example, the respective mechanical acoustic modifier may be housed within the respective back volume of the respective stem of the pair of stems 170. Each mechanical acoustic modifier may be comprised of acoustic dampening foam. The mechanical acoustic modifier may be positioned within the respective stem vertically above the respective cap (e.g., stem seal 504) where each cap defines the bottom end of the respective back volumes.

Thus, as shown in FIG. 5, an acoustic dampening foam 502 may be located within the first stem 170a, and specifically within the back volume 506. The acoustic dampening foam 502 is a type of mechanical acoustic modifier. In the example shown, the acoustic dampening foam 502 may span the curve of the first stem 170a, from the portion of the middle section 252 that is closest to the top end 514 to the portion of the top section 250 of the first stem 170a that is closest to the midline 503. The bottom end of the acoustic dampening foam 502 may be positioned towards the top of the middle section 252 of the first stem 170a. The top end of the acoustic dampening foam 502 may be positioned relatively horizontally with the top end nearer to the first speaker 117a than the bottom end of the acoustic dampening foam 502. As such, the acoustic dampening foam 502 may fill the inside of the top section 250 of the first stem 170a. The acoustic dampening foam 502 may not extend into the bottom section 254 of the first stem 170a. The acoustic dampening foam 502 may fill a suitable portion of the back volume 506, such as a third of the back volume, 25% of the back volume, 50% of the back volume, or another suitable portion. The portion of the back volume filled by the acoustic dampening foam 502 may be selected to provide the desired frequency response of the first speaker 117a.

The output tube 134, which surrounds the signal wires 302, may be mechanically isolated from the first stem 170a by the stem seal 504. The signal wires 302 may run the length of the output tube 134, and a first wire of the signal wires 302 may pass through the stem seal 504, traverse the length of the first stem 170a, including passing through the acoustic dampening foam 502, and connect to the first speaker 117a in order to transmit the audio signal from the chestpiece 110 to the first speaker 117a. As described previously, in a binaural configuration of the electronic stethoscope, the stems 170 include the first stem 170a and the second stem 170b. Consequently, the back volume 506 may correspond to the first stem 170a (and hence the left side of the headset 172) and there may be a symmetric back volume (not pictured) that corresponds to the second stem 170b (and hence the right side of the headset 172).

The amount of acoustic resistance provided by the acoustic dampening foam is controlled by the density and compression ratio of the foam after it has been assembled. The acoustic dampening foam may be an open cell foam that produces a tortuous path for air to flow through and increases friction. The acoustic dampening foam may be positioned at a region of a stem (e.g., the first stem 170a) where velocity of air within the stem is highest to achieve maximum dampening. At the resonant frequency (defined by the length of the stem), the velocity of the air within the stem may be highest close to the speaker driver and therefore the acoustic dampening foam 502 may be positioned at the curved region of the first stem 170a which is closer to the backside 520 of the speaker driver 510 than the middle section 252 of the first stem 170a. The acoustic dampening foam 502 may control the level and/or the overshoot of resonance and may dampen certain frequencies. The acoustic dampening foam 502 may alter the speed at which air pushed by the speaker moves, therefore altering the kinetic energy of the air. The amount of acoustic dampening foam 502 (e.g., the length of the foam within the stems 170) may control the level of resonance frequency and overshoot of resonance. The resonance frequency is the frequency at which a speaker naturally vibrates. This value determines the low frequency performance of a speaker. For sounds lower than the resonance frequency, the decibels of the speaker will remain constant when the ear tip is sealed in the listener's ear. At the resonant frequency the speaker will have variations in the sound pressure level output. By controlling the dampening of the resonance with the presence of the acoustic dampening foam, the variations of output can be minimized, thereby producing a flat frequency response down to low frequencies and reducing distortions.

The frequency response of a conventional acoustic stethoscope is in the low range, in some embodiments of a conventional acoustic stethoscope the frequency may be between 20-150 Hz. The frequency response of a conventional acoustic stethoscope is the target frequency response for the electronic stethoscope 100. The frequency response of the audio data from the speakers 117 may be tuned to the target frequency response through the use of the closed back volume 506 and the presence of the acoustic dampening foam 502. The acoustic dampening foam 502 and the stem seal 504 may be designed and positioned, as described above, to increase the low frequency volume response. A headphone or earbud that has an open back volume may be able to produce low frequency sounds, but the deviation required of the membrane of the speaker driver may cause distortion of the audio data. Because the acoustic dampening foam 502 and the back volume 506 may be designed to lower the resonance frequency and flatten the low-range frequency response, the electronic stethoscope 100 may closer mimic the frequency response that the user expects from a conventional acoustic stethoscope.

The frequency response that would be available with the signal wires 302 electrically transmitting the audio output of the chestpiece 110 to the speakers 117 without modification or a back volume would be higher than the expected range of frequencies provided by a conventional acoustic stethoscope and therefore less ideal for the user. The additions of the back volume 506 and the acoustic dampening foam 502 may collectively flatten the lower-range frequency response of the electronic stethoscope 100, allowing for the frequency response to closer mimic the expected frequency that a conventional acoustic stethoscope would provide so that the electronic stethoscope 100 may be used by the clinician without additional training.

Additionally, mechanically isolating the speakers 117 in the earpieces 116 from the output tube 134 may prevent transmission of sound artifacts from the output tube 134 to the user's ears. This may occur by minimizing ambient audio injected from the surroundings and minimizing artifact noise from touching the headset 172 as there is no acoustic connection from the air within the output tube 134 to the speakers 117.

Turning now to FIG. 6, a method 600 is shown for audio data transmission and conversion of an electronic stethoscope starting at a chestpiece and ending at earpieces of a headset. For example, the chestpiece herein described may be chestpiece 110 of the electronic stethoscope 100 shown in FIGS. 1A and 1B and the earpieces herein described may be earpieces 116 shown in FIGS. 1A, 2, 3, and 5. Further, components housed within the chestpiece may convert acoustic audio sounds (e.g., recorded physiological sounds) to electronic audio signals. For example, an electronic acoustic modifier herein described may be the electronic acoustic modifier 126 housed within the chestpiece 110 shown in FIG. 1B. Instructions for carrying out the method 600 may be executed by one or more processors, including a CPU of the electronic stethoscope (e.g., CPU 120 of FIG. 1B) based on instructions stored on a memory operatively coupled to each of the one or more processors (e.g., the memory 122 of FIG. 1B) and in conjunction with signals received from electronic components of the electronic stethoscope.

At 602, the method 600 includes transmitting an auscultated sound from a diaphragm of the chestpiece to inside the chestpiece of the electronic stethoscope. The auscultated sounds, or recorded physiological sounds may be derived from acoustic pressure waves from physiological sounds of a patient of which the stethoscope chestpiece is placed directly on. The recorded physiological sounds may be heart sounds, lung sounds, bowel sounds, fistula sounds, or any other measurable sound made by the patient's body. In order for the stethoscope chestpiece to generate the auscultated sounds, the electronic stethoscope (specifically the stethoscope chestpiece component of the electronic stethoscope) may be placed on the patient (e.g., the patient 180 shown in FIG. 1A).

Once the stethoscope chestpiece is placed on the patient's body, the auscultated sounds may be then transmitted from the diaphragm to inside the chestpiece. The diaphragm of the chestpiece may be in acoustic communication with the interior of the chestpiece. The auscultated sound that is transmitted from the diaphragm to the chestpiece may therefore be a physiological sound.

At 604, the method 600 includes transmitting the auscultated sound to a microphone. The auscultated sound is transmitted for detection by the microphone (e.g., microphone 128 shown in FIG. 1B) for conversion to an auscultated electronic signal. The microphone may be housed within the chestpiece body along with other components including, in some embodiments, the CPU, the memory, and a battery. The microphone may be powered by the battery and may be in electrical communication with the other electrical components of the chestpiece and the headset of the electronic stethoscope.

At 606, the method 600 includes converting the auscultated sound to an auscultated electronic signal. The auscultated sound has been transmitted from the diaphragm to the inside of the chestpiece that houses the microphone, as described at 604 and 606. The microphone may then convert the sound to an electronic signal. In some embodiments, there may be a second microphone that detects noise from the surrounding environment. The second microphone may then convert the noise detected from the environment to an electric signal. The detected noise from the environment, in combination with the sounds detected by the microphone, may be used in active noise cancellation processes.

At 608, the method 600 includes optionally transmitting the auscultated electronic signal from the microphone to an electronic acoustic modifier. The electronic acoustic modifier (e.g., electronic acoustic modifier 126 shown in FIG. 1B) may be housed within the same chestpiece as the microphone.

At 610, the method 600 includes modifying the auscultated electronic signal to form a modified electronic signal. After the auscultated electronic signal is transmitted from the microphone to the electronic acoustic modifier, the electronic acoustic modifier (e.g., electronic acoustic modifier 126 shown in FIG. 1B) may modify the auscultated electronic signal into a modified electronic signal that can be further transmitted towards an output. The electronic acoustic modifier may be configured to receive the auscultated electronic signal from the microphone, modify the auscultated electronic signal to form a modified electronic signal, and transmit the modified electronic signal towards the output. The electronic acoustic modifier may, in some examples, include an amplifier to amplify the auscultated electronic signal prior to transmission to the speakers. Alternatively, a separate amplifier may be housed within the chestpiece. Also alternatively, in some examples, the auscultated signal from the microphone may be transmitted directly to speakers. In such examples, modification may not be performed on the auscultated electronic signal outputted from the microphone, though a separate amplifier may be positioned within the chestpiece or the headset.

At 612, the method 600 includes transmitting the modified electronic signal from the electronic acoustic modifier to signal wires. As described previously, when the electrical connection between the chestpiece and the headset is in place, the electronic acoustic modifier may transmit the modified electronic signal to signal wires. The signal wires (e.g., signal wires 302 shown in FIG. 3) may be housed within an output tube (e.g., output tube 134) of the headset. The signal wires may run the length of the output tube and then pass through stem seal(s) (e.g., stem seal 504 shown in FIG. 5), for example cap(s) or plug(s), that mechanically isolate the output tube from stem(s) (e.g., first stem 170a shown in FIG. 5) of the headset. The stem seal(s) may be positioned at the distal portion of the stem(s). The signal wires may then run the length of the stem(s) and electronically connect to speaker(s) housed inside earpiece(s). The electronic stethoscope may be a binaural configuration, meaning that the signal wires may branch into two sides (which each feed one of the speakers (left or right)) at the level of the output tube prior to the stem seal.

At 614, the method 600 includes transmitting the modified electronic signal through the output tube and the stem(s) via the signal wires. As discussed above, the signal wires traverse a path through the output tube and the stem(s) in order to connect to the speaker(s) housed within the earpiece(s). The modified electronic signal that has been transmitted from the electronic acoustic modifier to the signal wires may be transmitted down the length of the signal wires. As described above, the signal wires are housed within the output tube and the stem(s), passing through the stem seal(s) as it transitions from being within the output tube to the stem(s). Consequently, the modified electronic signal may be transmitted through the output tube and into the stem via the signal wires. The stem seal(s) also define back volume(s) for the speaker(s) within the stem(s). The back volume(s) of air defines the air that the speaker(s) may push against when transmitting audio out of the earpiece(s). The stem(s) also house acoustic dampening foam nearer to the speaker(s) than the stem seal's position is. The signal wires that are transmitting the modified electronic signal may also pass through the acoustic dampening foam in addition to passing through the steam seal(s). The acoustic dampening foam (e.g., acoustic dampening foam 502 shown in FIG. 5) may be present to further modify the sound produced by the speaker(s) as the speaker(s) pushes against the air in the back volume.

At 616, the method 600 includes transmitting the modified electronic signal to the speakers. The signal wires have transmitted the modified electronic signal from the electronic acoustic modifier down the length of the output tube and the stem. The signal wires then electrically connect to the speakers. As previously described, when the electronic stethoscope is in the assembled state, electrical connections are established between the electrical components of the electronic stethoscope and those components are powered on. Such components include both the signal wires and the speakers. The modified electronic signal may be transmitted from the signal wires to the speakers due to the electrical connection between the two.

At 618, the method 600 includes converting the modified electronic signal to a modified sound by the speaker(s). The speaker(s) (e.g., speakers 117 shown in FIGS. 1A and 3) may be configured to receive the modified electronic signal from the signal wires and to convert the modified electronic signal to modified sound for transmission to the earpieces. The back volume defined by the dimensions of the stem behind the speaker may flatten the frequency response in the low-frequency range and prevent the speaker from being overdriven and may adjust the resonance frequency to the desired frequency. The acoustic dampening foam positioned within the stem behind the speaker(s) may control the level of the resonance frequency and minimize overshoot of the resonance frequency.

At 620, the method 600 includes transmitting the modified sound from the speakers to the earpieces for sound output. The modified sound, which has been converted from a modified electronic signal by the speakers, may be transmitted from the speakers to the output of the earpieces (e.g., projected to a listener). The earpieces may then output the modified sound into the user's ears (e.g., the clinician's ears) during conditions in which the earpieces are positioned in the ears of the user. The method 600 may then end.

The technical effect of providing a closed back volume within a stem of a headset for a speaker housed at a distal end of the headset and configured to be positioned in an ear of a listener is that a target range of frequencies may be accurately projected by the speaker while minimizing unwanted noise.

In another representation, a method for an electronic stethoscope includes recording physiological sounds of a patient with a microphone positioned within a chestpiece of the electronic stethoscope, transmitting, via signal wires of the electronic stethoscope, the recorded physiological sounds to a pair of speakers coupled at respective ends of a headset of the electronic stethoscope, and projecting the recorded physiological sounds to a listener via the pair of speakers, wherein the pair of speakers project the recorded physiological sounds with a frequency response that is tuned at least in part by a pair of back volumes housed within the headset.

In another representation, an article of manufacture includes a headset including a speaker to project audio data generated by a chestpiece, the headset including a connector configured to couple the chestpiece, the connector comprising a bayonet fitting configured to couple the chestpiece to an output tube of the headset and enable electrical connection between the chestpiece and signal wires housed in the output tube via a jack plug of the headset.

The disclosure also provides support for an article of manufacture, comprising: a headset including a speaker to project audio data generated by a chestpiece, the headset defining a closed back volume for the speaker. In a first example of the article, the back volume comprises a tube of the headset with a volume tuned to provide a target frequency response for the speaker. In a second example of the article, optionally including the first example, the headset comprises an output tube configured to couple to the chestpiece and a first stem and a second stem branching from the output tube, the first stem coupled to a first earpiece housing the speaker, and wherein the tube of the headset is the first stem. In a third example of the article, optionally including one or both of the first and second examples, the first stem includes a cap to seal the back volume from the output tube. In a fourth example of the article, optionally including one or more or each of the first through third examples, the cap is positioned within the first stem at a location selected to form the volume. In a fifth example of the article, optionally including one or more or each of the first through fourth examples, the speaker is a first speaker and the closed back volume is a first closed back volume, and wherein the second stem is coupled to a second earpiece housing a second speaker, and wherein the second stem forms a second closed back volume for the second speaker. In a sixth example of the article, optionally including one or more or each of the first through fifth examples, the closed back volume includes a mechanical acoustic modifier. In a seventh example of the article, optionally including one or more or each of the first through sixth examples, the mechanical acoustic modifier comprises acoustic dampening foam. In an eighth example of the article, optionally including one or more or each of the first through seventh examples, target frequency response includes frequencies in a range of 20-150 Hz. In a ninth example of the article, optionally include one or more or each of the first through eighth examples, the article of manufacture comprises an electronic stethoscope.

The disclosure also provides support for an electronic stethoscope, comprising: a headset including a speaker to project audio data generated by a chestpiece and signal wires configured to transmit the audio data from the chestpiece to the speaker, the headset defining a closed back volume for the speaker. In a first example of the stethoscope, the back volume comprises a tube of the headset with a volume tuned to provide a target frequency response for the speaker. In a second example of the stethoscope, optionally including the first example, the headset comprises an output tube configured to couple to the chestpiece and a first stem and a second stem branching from the output tube, the first stem coupled to a first earpiece housing the speaker, and wherein the tube of the headset is the first stem. In a third example of the stethoscope, optionally including one or both of the first and second examples, the first stem includes a cap to seal the back volume from the output tube. In a fourth example of the stethoscope, optionally including one or more or each of the first through third examples, the cap is positioned within the first stem at a location selected to form the volume. In a fifth example of the stethoscope, optionally including one or more or each of the first through fourth examples, the speaker is a first speaker and the closed back volume is a first closed back volume, and wherein the second stem is coupled to a second earpiece housing a second speaker, and wherein the second stem forms a second closed back volume for the second speaker. In a sixth example of the stethoscope, optionally including one or more or each of the first through fifth examples, the closed back volume includes a mechanical acoustic modifier. In a seventh example of the stethoscope, optionally including one or more or each of the first through sixth examples, the mechanical acoustic modifier comprises acoustic dampening foam. In an eighth example of the stethoscope, optionally including one or more or each of the first through seventh examples, target frequency response includes low audio frequencies typically produced by the heart and lungs. In a ninth example of the stethoscope, optionally including one or more or each of the first through eighth examples, the stethoscope further comprises: a connection mechanism configured to couple headset to the chestpiece, the connection mechanism including a twist-lock mechanism which holds the headset rigidly against the chestpiece and provides electrical contact. In a tenth example of the stethoscope, optionally including one or more or each of the first through ninth examples, the connection mechanism is releasable. In an eleventh example of the stethoscope, optionally including one or more or each of the first through tenth examples, the twist-lock mechanism is configured to lock with less than one full rotation. In a twelfth example of the stethoscope, optionally including one or more or each of the first through eleventh examples, the connection mechanism, when locked, is configured to hold the headset at a fixed orientation relative to the chestpiece.

The disclosure also provides support for an electronic stethoscope, comprising: a chestpiece operable to generate audio data of a patient, and a headset including an output tube and a first stem and a second stem branching from the output tube, the first stem coupled to a first earpiece including a first speaker, the headset further including signal wires configured to transmit the audio data from the chestpiece to the first speaker, the first speaker including a speaker driver having a backside exposed to a closed back volume formed by the first earpiece and a hollow region within the first stem. In a first example of the system, the hollow region within the first stem that forms the closed back volume terminates at a cap within the first stem, the cap positioned at a location selected to provide a target frequency response for the first speaker. In a second example of the system, optionally including the first example, the hollow region within the first stem that forms the closed back volume includes a mechanical acoustic modifier. In a third example of the system, optionally including one or both of the first and second examples, the headset includes a connector configured to be housed within a corresponding connector of the chestpiece. In a fourth example of the system, optionally including one or more or each of the first through third examples, the second stem is coupled to a second earpiece including a second speaker, the second speaker including a second speaker driver having a backside exposed to a second closed back volume formed by the second earpiece and a second hollow region within the second stem.

As used herein, an element or step recited in the singular and preceded with the word “a” or “an” should be understood as not excluding plural of said elements or steps, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a particular property may include additional such elements not having that property. The terms “including” and “in which” are used as the plain-language equivalents of the respective terms “comprising” and “wherein.” Moreover, the terms “first,” “second,” and “third,” etc. are used merely as labels, and are not intended to impose numerical requirements or a particular positional order on their objects.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. An electronic stethoscope, comprising:
a headset including a speaker to project audio data generated by a chestpiece and signal wires configured to transmit the audio data from the chestpiece to the speaker, the headset defining a closed back volume for the speaker.

2. The electronic stethoscope of claim 1, wherein the back volume comprises a tube of the headset with a volume tuned to provide a target frequency response for the speaker.

3. The electronic stethoscope of claim 2, wherein the headset comprises an output tube configured to couple to the chestpiece and a first stem and a second stem branching from the output tube, the first stem coupled to a first earpiece housing the speaker, and wherein the tube of the headset is the first stem.

4. The electronic stethoscope of claim 3, wherein the first stem includes a cap to seal the back volume from the output tube.

5. The electronic stethoscope of claim 4, wherein the cap is positioned within the first stem at a location selected to form the volume.

6. The electronic stethoscope of claim 3, wherein the speaker is a first speaker and the closed back volume is a first closed back volume, and wherein the second stem is coupled to a second earpiece housing a second speaker, and wherein the second stem forms a second closed back volume for the second speaker.

7. The electronic stethoscope of claim 2, wherein the closed back volume includes a mechanical acoustic modifier.

8. The electronic stethoscope of claim 7, wherein the mechanical acoustic modifier comprises acoustic dampening foam.

9. The electronic stethoscope of claim 2, wherein target frequency response includes low audio frequencies typically produced by the heart and lungs.

10. The electronic stethoscope of claim 1, further comprising a connection mechanism configured to couple headset to the chestpiece, the connection mechanism including a twist-lock mechanism which holds the headset rigidly against the chestpiece and provides electrical contact.

11. The electronic stethoscope of claim 10, wherein the connection mechanism is releasable.

12. The electronic stethoscope of claim 10, wherein the twist-lock mechanism is configured to lock with less than one full rotation.

13. The electronic stethoscope of claim 10, wherein the connection mechanism, when locked, is configured to hold the headset at a fixed orientation relative to the chestpiece.

14. An electronic stethoscope, comprising:
a chestpiece operable to generate audio data of a patient; and
a headset including an output tube and a first stem and a second stem branching from the output tube, the first stem coupled to a first earpiece including a first speaker, the headset further including signal wires configured to transmit the audio data from the chestpiece to the first speaker, the first speaker including a speaker driver having a backside exposed to a closed back volume formed by the first earpiece and a hollow region within the first stem.

15. The electronic stethoscope of claim 14, wherein the hollow region within the first stem that forms the closed back volume terminates at a cap within the first stem, the cap positioned at a location selected to provide a target frequency response for the first speaker.

16. The electronic stethoscope of claim 14, wherein the hollow region within the first stem that forms the closed back volume includes a mechanical acoustic modifier.

17. The electronic stethoscope of claim 14, wherein the headset includes a connector configured to be housed within a corresponding connector of the chestpiece.

18. The electronic stethoscope of claim 14, wherein the second stem is coupled to a second earpiece including a second speaker, the second speaker including a second speaker driver having a backside exposed to a second closed back volume formed by the second earpiece and a second hollow region within the second stem.
